SEQUENCE LISTING (1 sequence)

SEQ ID NO: 1 - RNA, 1367 nt, Lactobacillus sakei
  source 1..1367: mol_type = rRNA
gcgtgcctaatacatgcaagtcgaacgcactctcgtttagattgaaggagcttgctcctgattgataaacatttgagtgagtggcggacgggtgagtaacacgtgggtaacctgccctaaagtgggggataacatttggaaacagatgctaataccgcataaaacctaacaccgcatggtgtagggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaatgtatctgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagccggaatcgctagtaatcgcggatcagcatgccgcggtgaatac

(19) United States
(12) Patent Application Publication
(10) Pub. No.: US 2025/0032562 A1
(43) Pub. Date: Jan. 30, 2025

(54) NOVEL LACTOBACILLUS SAKEI STRAIN AND USE THEREOF

(71) Applicant: LG H & H CO., LTD., Seoul (KR)

(72) Inventors: Jungha CHOO, Seoul (KR); Minjung KWON, Seoul (KR); So Young LEE, Seoul (KR); Sunghwan HWANG, Seoul (KR)

(73) Assignee: LG H & H CO., LTD., Seoul (KR)

(21) Appl. No.: 18/781,747

(22) Filed: Jul. 23, 2024

(30) Foreign Application Priority Data
Jul. 24, 2023 (KR) 10-2023-0096239

(51) Int. Cl. A61K 35/747; A61K 8/99; A61P 17/00; A61Q 19/00; A61Q 19/02; A61Q 19/08

(52) U.S. Cl. CPC A61K 35/747; A61K 8/99; A61P 17/00; A61Q 19/007; A61Q 19/02; A61Q 19/08

(57) ABSTRACT

Provided are a Lactobacillus sakei LGHNH22021 strain; a cosmetic composition, a quasi-drug composition, and a food composition, each composition for skin improvement including any one or more of the strain, a culture product of the strain, a fermentation product of the strain, a culture medium of the strain, an extract of the strain, and a crushed product of the strain; a composition for promoting the proliferation of Staphylococcus epidermidis which is a beneficial skin bacterium, the composition including the strain; a composition for inhibiting the proliferation of Staphylococcus aureus which is a harmful skin bacterium, the composition including the strain; and a method for preparing a fermentation product or a lysate, the method including fermenting or crushing the strain.

### REFERENCE TO ELECTRONIC SEQUENCE LISTING

The application contains a Sequence Listing which has been submitted electronically in .XML format and is hereby incorporated by reference in its entirety. Said .XML copy, created on May 20, 2024, is named “OPA23311_Seqeunce Listing.xml” and is 4,096 bytes in size. The sequence listing contained in this .XML file is part of the specification and is hereby incorporated by reference herein in its entirety.

### TECHNICAL FIELD

The present disclosure relates to a Lactobacillus sakei LGHNH22021 strain; a cosmetic composition, a quasi-drug composition, and a food composition, each composition for skin improvement including any one or more of the strain, a culture product of the strain, a fermentation product of the strain, a culture medium of the strain, an extract of the strain, and a crushed product of the strain; a composition for promoting the proliferation of Staphylococcus epidermidis which is a beneficial skin bacterium, the composition including the strain; a composition for inhibiting the proliferation of Staphylococcus aureus which is a harmful skin bacterium, the composition including the strain; and a method for preparing a fermentation product or a lysate, the method including fermenting or crushing the strain.

### BACKGROUND ART

Various symbiotic microorganisms exist in living organisms. Among these symbiotic microorganisms, some produce lactic acid as a metabolic product while growing. These bacteria are collectively called lactic acid bacteria and are widely distributed in the natural world, including in various organs of humans or mammals, such as the digestive tract, oral cavity, vagina, etc., as well as in plants, various fermented foods, soil, etc. These lactic acid bacteria are microorganisms that have been used by humans for a long time and have been used in many different fermented foods (fermented dairy products, fermented foods, sauces, kimchi, etc.). Recently, interest in the efficacy of lactic acid bacteria has been increasing, and research is being actively conducted on the use of lactic acid bacteria not only in food materials but also in cosmetics and pharmaceuticals.

Land plant-based materials used in cosmetics, quasi-drugs, etc. require a lot of cost and time to cultivate, whereas microorganisms are highly sustainable materials that can be permanently cultured with a single acquisition. In addition, since the characteristics and efficacy of biological resources of the same species vary depending on the strain and origin, it is necessary to discover as many biological resources as possible and to utilize them according to their characteristics.

In this regard, research has been conducted on the use of Lactobacillus plantarum strain (KR 10-2022-0081475 A) and Lactobacillus fermentum strain (KR 10-2022-0170253 A) belonging to the genus Lactobacillus as raw materials for cosmetics. However, the industrial applications of lactic acid bacteria are still extremely limited in terms of scale and diversity compared to the abundance of species, and thus it is necessary to expand the field of application.

Under this background, the present inventors have conducted research on lactic acid bacteria from natural resources, which are safer and have excellent skin improvement effects, and they found that a novel Lactobacillus sakei LGHNH22021 strain has significantly superior skin improvement effects, thereby completing the present disclosure.

### DISCLOSURE

### Technical Problem

An object of the present disclosure is to provide a Lactobacillus sakei LGHNH22021 strain.

Another object of the present disclosure is to provide a cosmetic composition for skin improvement, the cosmetic composition including any one or more of the strain, a culture product of the strain, a fermentation product of the strain, a culture medium of the strain, an extract of the strain, and a crushed product of the strain.

Still another object of the present disclosure is to provide a quasi-drug composition for skin improvement, the quasi-drug composition including any one or more of the strain, a culture product of the strain, a fermentation product of the strain, a culture medium of the strain, an extract of the strain, and a crushed product of the strain.

Still another object of the present disclosure is to provide a food composition for skin improvement, the food composition including any one or more of the strain, a culture product of the strain, a fermentation product of the strain, a culture medium of the strain, an extract of the strain, and a crushed product of the strain.

Still another object of the present disclosure is to provide a composition for promoting the proliferation of Staphylococcus epidermidis which is a beneficial skin bacterium, the composition including any one or more of the strain, a culture product of the strain, a fermentation product of the strain, a culture medium of the strain, an extract of the strain, and a crushed product of the strain.

Still another object of the present disclosure is to provide a composition for inhibiting the proliferation of Staphylococcus aureus which is a harmful skin bacterium, the composition including any one or more of the strain, a culture product of the strain, a fermentation product of the strain, a culture medium of the strain, an extract of the strain, and a crushed product of the strain.

Still another object of the present disclosure is to provide a method for preparing a fermentation product, the method including fermenting the strain.

Still another object of the present disclosure is to provide a method for preparing a lysate, the method including crushing the strain.

Still another object of the present disclosure is to provide a method for improving skin conditions, the method including applying to, a subject' skin, a composition including any one or more of the strain, a culture product of the strain, a fermentation product of the strain, a culture medium of the strain, an extract of the strain, and a crushed product of the strain.

Still another object of the present disclosure is to provide use of a composition including any one or more of the strain, a culture product of the strain, a fermentation product of the strain, a culture medium of the strain, an extract of the strain, and a crushed product of the strain in improving skin conditions.

### Technical Solution

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Further, those skilled in the art will recognize, or be able to ascertain using no more than routine experimentation, many equivalents to the specific embodiments of the disclosure described herein. Further, these equivalents should be interpreted to fall within the present disclosure.

An aspect of the present disclosure provides a Lactobacillus sakei LGHNH22021 strain which is deposited with Accession No. KCTC 15087BP.

In one specific embodiment, the strain may have one or more characteristics selected from the group consisting of anti-aging, wrinkle improvement, elasticity enhancement, skin regeneration, skin whitening, skin moisturization, skin soothing, anti-inflammation, and skin microbiome enhancement, but is not limited thereto.

As used herein, the term “Lactobacillus sakei” is an aerobic or facultative anaerobic microorganism belonging to Gram-positive bacillus that is widely distributed in nature.

The present inventors have developed a novel strain belonging to Lactobacillus sakei, which was named Lactobacillus sakei LGHNH22021 and deposited with Accession No. KCTC 15087BP.

Specifically, the novel Lactobacillus sakei LGHNH22021 strain of the present disclosure is a core-microbiome of Achillea millefolium, and may be isolated and obtained from Achillea millefolium, but is not limited thereto, as long as it has the same characteristic as the deposited strain Lactobacillus sakei LGHNH22021.

In the present disclosure, a method of isolating the strain from Achillea millefolium is not particularly limited, and a strain having the same characteristics as the deposited strain may be isolated according to a method commonly used in the relevant technical field or a similar field.

As used herein, the term “strain” refers to a population of individuals that originate from a single cell and proliferate through asexual reproduction to have the same genetic characteristics. Within a species, there are several strains (genetic variants) with different genetic characteristics. In the present disclosure, the strain refers to Lactobacillus sakei LGHNH22021, and specifically, Lactobacillus sakei LGHNH22021 deposited with Accession No. KCTC 15087BP, but is not limited thereto.

In the present disclosure, the strain may include a cell of the Lactobacillus sakei LGHNH22021 strain, a dried cell, etc. In this regard, the dried cell may be a dried bacterium such as a spray-dried bacterium, a freeze-dried bacterium, a vacuum-dried bacterium, or a drum-dried bacterium, etc.

Another aspect of the present disclosure provides a cosmetic composition for skin improvement, the cosmetic composition including any one or more of the Lactobacillus sakei LGHNH22021 strain, a culture product of the strain, a fermentation product of the strain, a culture medium of the strain, an extract of the strain, and a crushed product of the strain.

Specifically, the present inventors confirmed that Lactobacillus sakei LGHNH22021 strain derived from Achillea millefolium has excellent skin improvement effects.

As used herein, the term “culture product” refers to a product obtained by culturing the above strain in a medium. For example, the culture product of the present disclosure may include components remaining in the medium after harvesting the strain from the culture medium of Lactobacillus sakei LGHNH22021, or may include components of the culture medium containing the strain.

As used herein, the term “culture medium” refers to a part of the above culture product, excluding the strain. For example, the culture medium of the present disclosure may include a culture supernatant excluding the above strain after centrifugation of the culture medium of Lactobacillus sakei LGHNH22021, or a part of the culture supernatant, excluding the crushed product.

The culture product may be the Lactobacillus sakei LGHNH22021 strain, the entire culture product of the strain, the culture medium, fractions thereof, etc. The culture medium may be a culture supernatant of the Lactobacillus sakei LGHNH22021 strain, etc. In this regard, the culture supernatant may be obtained by centrifuging the culture product of the strain, and the fraction may be obtained by subjecting the strain, the culture product, and the culture supernatant to methods such as centrifugation, chromatography, etc.

As for the media and other culture conditions used for culturing the strain of the present disclosure, any medium that is used for usual culture of the microorganisms of the genus Lactobacillus may be used without particular limitation. Specifically, the strain of the present disclosure may be cultured under aerobic or anaerobic conditions in a common medium containing appropriate carbon sources, nitrogen sources, phosphorus sources, inorganic compounds, amino acids, and/or vitamins, while controlling the temperature, pH, etc.

In the present disclosure, the carbon source may include carbohydrates, such as glucose, fructose, sucrose, maltose, etc.; sugar alcohols, such as mannitol, sorbitol, etc.; organic acids, such as pyruvic acid, lactic acid, citric acid, etc.; and amino acids, such as glutamate, methionine, lysine, etc., but is not limited thereto. In addition, natural organic nutrient sources may be used, such as starch hydrolysates, molasses, blackstrap molasses, rice bran, cassava, bagasse, and corn steep liquor, and carbohydrates, such as glucose and sterile pretreated molasses (i.e., molasses converted to reduced sugars) may be used, and appropriate amounts of various other carbon sources may be used without limitation. These carbon sources may be used alone or in a combination of two or more thereof, but are not limited thereto.

As for the nitrogen sources, inorganic nitrogen sources, such as ammonia, ammonium sulfate, ammonium chloride, ammonium acetate, ammonium phosphate, ammonium carbonate, ammonium nitrate, etc.; amino acids, and organic nitrogen sources, such as peptone, NZ-amine, meat extracts, yeast extracts, malt extracts, corn steep liquor, casein hydrolysates, fishes or decomposition products thereof, defatted soybean cake or degradation products thereof, etc. may be used. These nitrogen sources may be used alone or in a combination of two or more thereof, but are not limited thereto.

The phosphorus sources may include potassium phosphate monobasic, potassium phosphate dibasic, and sodium-containing salts corresponding thereto. As for inorganic compounds, sodium chloride, calcium chloride, iron chloride, magnesium sulfate, iron sulfate, manganese sulfate, calcium carbonate, etc. may be used.

In addition, amino acids, vitamins, and/or suitable precursors may be included in the medium. Specifically, L-amino acids, etc. may be added to the culture medium of the strain. Specifically, glycine, glutamate, and/or cysteine, etc. may be added, and as needed, L-amino acids such as lysine, etc. may be further added, but are not necessarily limited thereto.

These media or precursors may be added to the culture product or culture medium in a batch or continuous manner, but are not limited thereto.

In the present disclosure, the pH of the culture product or culture medium may be adjusted by adding compounds, such as ammonium hydroxide, potassium hydroxide, ammonia, phosphoric acid, and sulfuric acid, etc., to the culture product or culture medium in an appropriate manner during the culture of the strain. In addition, an anti-foaming agent, such as a fatty acid polyglycol ester, may be used to suppress foam formation during the culture. In addition, oxygen or oxygen-containing gas may be injected into the culture product or culture medium to maintain the aerobic state of the culture product or culture medium, or no gas may be injected or nitrogen, hydrogen or carbon dioxide gas may be injected to maintain the anaerobic or non-aerobic state.

As used herein, the term “fermentation product” may include a medium including a strain obtained by culturing a microorganism in the medium for a predetermined period of time, metabolites thereof, extra nutrients, etc., a culture medium obtained by removing the strain from the medium, a dilution or concentrate of the medium or culture medium, or a dried product thereof. The term “fermentation” refers to a process in which microorganisms decompose organic matter using their own enzymes, excluding a putrefaction reaction. In the present disclosure, a method of obtaining the fermentation product from the strain is not particularly limited, and the fermentation product may be obtained according to a method commonly used in the relevant technical field or a similar field.

In the present disclosure, as for the method of obtaining the fermentation product, it may be obtained by treating the separated strain with sugar, which is an energy source of the strain, and then fermenting the same. However, the temperature and fermentation time of the fermentation process are not particularly limited, and may be selected in various ways depending on fermentation precursor materials, fermentation conditions, the type of substance desired to be obtained from the fermentation product, etc.

In the present disclosure, the fermentation process may be performed at a temperature of specifically 20° C. to 45° C., more specifically 25° C. to 35° C., and much more specifically 27° C. to 33° C., and the fermentation may be performed for a period of specifically 1 day to 14 days, more specifically 1 day to 3 days, and much more specifically 1 day to 2 days.

In the present disclosure, the fermentation product obtained from the strain includes all kinds of materials including the fermentation product generated from the strain, such as not only a fermented material itself but also a culture medium of the strain in which the strain and the culture product coexist, a fermentation product obtained by filtering the strain from the culture medium, a supernatant obtained by centrifugation of the culture medium, excluding the strain, a fermentation product obtained by sterilizing the strain and filtering the same from the culture medium, an extract obtained by extracting the fermentation product or the culture medium containing the same, a dilution obtained by diluting the fermentation product or the extract thereof, a dried product obtained by drying the fermentation product or the extract thereof, a lysate obtained by collecting cells of the strain and disrupting the same, etc.

In the present disclosure, with regard to the concentration of Lactobacillus sakei LGHNH22021 fermentation product, % by weight of the Lactobacillus sakei LGHNH22021 strain may be 0.000001% to 100%, 0.000001% to 50%, 0.000001% to 40%, 0.000001% to 30%, 0.000001% to 20%, 0.000001% to 15%, 0.000001% to 10%, 0.00001% to 30%, 0.00001% to 20%, 0.00001% to 15%, 0.00001% to 10%, 0.0001% to 30%, 0.0001% to 20%, 0.0001% to 15%, 0.0001% to 10%, 0.001% to 30%, 0.001% to 20%, 0.001% to 15%, 0.001% to 10%, 0.01% to 30%, 0.01% to 20%, 0.01% to 15%, 0.01% to 10%, 0.1% to 30%, 0.1% to 20%, 0.1% to 15%, 0.1% to 10%, 1% to 30%, 1% to 20%, 1% to 15%, or 1% to 10%, based on the total % by weight of the composition, but is not limited thereto.

As used herein, the term “extract” refers to a resulting product such as liquid components obtained by immersing a target material in various solvents and then extracting the same at room temperature, low temperature, or warm temperature for a predetermined period of time, and solid components obtained by removing the solvent from the liquid components, etc. In addition to the resulting product, the extract may be comprehensively interpreted as including all dilutions of the above resulting product, concentrates thereof, crude products thereof, purified products thereof, etc. Accordingly, the Lactobacillus sakei LGHNH22021 extract provided in the present disclosure may be interpreted as including a liquid extract itself and all formulations of the extract which may be formed using the liquid extract, such as a liquid extract obtained by extracting the same, a diluted or concentrated liquid of the liquid extract, a dried product obtained by drying the liquid extract, a crude product or purified product of the liquid extract, or a mixture thereof, etc.

A method of extracting the Lactobacillus sakei LGHNH22021 of the present disclosure is not particularly limited, and may be extracted according to a method commonly used in the art. Non-limiting examples of the extraction method may include hot water extraction, ultrasonic extraction, filtration, and reflux extraction, etc., which may be performed alone or in combination of two or more thereof.

The type of solvent used for the extraction is not particularly limited, and any solvent known in the art may be used. Non-limiting examples of the extraction solvent may include water, C1 to C4 lower alcohols, ether, ethyl acetate, glycerin, butylene glycol, propylene glycol, methyl acetate, ethyl acetate, chloroform, acetone, benzene, hexane, diethyl ether, dichloromethane, or mixed solvents thereof, which may be used alone or in combination of two or more thereof. Specifically, water may be used. When alcohol is used as the solvent, C1 to C4 alcohol may be specifically used.

In the present disclosure, the extract may be an extract of the culture product, fermentation product, culture medium, crushed product, or strain (pellet), and specifically, an extract obtained by centrifuging the culture product and then concentrating the strain, from which the supernant is removed, under reduced pressure, but is not limited thereto.

In the present disclosure, with regard to the concentration of Lactobacillus sakei LGHNH22021 extract, % by weight of the Lactobacillus sakei LGHNH22021 strain may be 0.000001% to 100%, 0.000001% to 50%, 0.000001% to 40%, 0.000001% to 30%, 0.000001% to 20%, 0.000001% to 15%, 0.000001% to 10%, 0.00001% to 30%, 0.00001% to 20%, 0.00001% to 15%, 0.00001% to 10%, 0.0001% to 30%, 0.0001% to 20%, 0.0001% to 15%, 0.0001% to 10%, 0.001% to 30%, 0.001% to 20%, 0.001% to 15%, 0.001% to 10%, 0.01% to 30%, 0.01% to 20%, 0.01% to 15%, 0.01% to 10%, 0.1% to 30%, 0.1% to 20%, 0.1% to 15%, 0.1% to 10%, 1% to 30%, 1% to 20%, 1% to 15%, or 1% to 10%, based on the total % by weight of the composition, but is not limited thereto.

As used herein, the term “crushed product (or lysate)” refers to a product obtained by physical or chemical treatment of the strain itself, high-pressure sterilization or ultrasonic treatment thereof, or by crushing the supernatant which is obtained by centrifugation of the culture product of the strain, fermentation product or culture medium, but is not limited thereto. The crushed product may be used interchangeably with the term “lysate.”

The composition including the Lactobacillus sakei LGHNH22021 strain deposited with KCTC 15087BP of the present disclosure, the culture product, fermentation product, culture medium, extract, or crushed product of the strain, or a combination thereof has excellent skin improvement effect, as compared to a composition including another Lactobacillus sakei LGHNH22021 strain, a culture product, fermentation product, culture medium, extract, or crushed product thereof, etc.

In the present disclosure, the “skin improvement” refers to any action that at least reduces parameters related to the alleviation or treatment of skin conditions, e.g., the severity of symptoms. For example, the skin improvement may be one or more selected from the group consisting of anti-aging, wrinkle improvement, elasticity enhancement, skin regeneration, skin whitening, skin moisturization, skin soothing, anti-inflammation, and skin microbiome enhancement, but is not limited thereto.

As used herein, the term “improvement” refers to any action that at least reduces parameters related to the alleviation or treatment of conditions, e.g., the severity of symptoms.

The skin improvement may be achieved through one or more selected from the group consisting of promoting collagen synthesis, promoting keratinocyte growth, inhibiting melanin synthesis, increasing hyaluronic acid production, suppressing NO production, increasing the growth of beneficial bacteria and inhibiting the growth of harmful bacteria, but is not limited thereto.

As used herein, the term “aging” refers to a phenomenon of decline in biological structure and function over time, and the “anti-aging” of the present disclosure is not limited to the extent as long as aging is improved, such as prevention, suppression, or delay of aging. The anti-aging may include skin elasticity improvement, skin regeneration, and wrinkle improvement, etc., but is not limited thereto.

As used herein, the term “skin wrinkle” refers to a phenomenon in which skin folds are caused by loss of skin elasticity due to skin aging, and the “wrinkle improvement” of the present disclosure refers to suppressing or inhibiting the formation of wrinkles on the skin or alleviating wrinkles that have already formed, but is not limited thereto.

As used herein, the term “skin elasticity” refers to the ability to keep the skin tight when pressed with a finger, and the “elasticity improvement” or “elasticity enhancement” refers to increasing the skin elasticity by strengthening an individual's subcutaneous fat layer structure, and may mean increasing skin elasticity using the composition of the present disclosure. Specifically, the anti-aging and elasticity improvement may be due to stimulation of collagen synthesis, but is not limited thereto.

As used herein, the term “collagen” is a fibrous protein abundantly found in most animals, particularly, in mammals, and makes up most of all connective tissue in the body, including skin, cartilage, etc. Fibroblasts, which are the most common cells in the body, produce and release collagen. Gelatin, which is widely used in cooking, or food and pharmaceutical industries, is an irreversibly hydrolyzed form of collagen. Since collagen reduction is the main cause of wrinkles and reduced skin elasticity, collagen synthesis is essential for improving wrinkles and enhancing elasticity.

In the present disclosure, the promotion of collagen synthesis may be due to the Lactobacillus sakei LGHNH22021 strain, the culture product of the strain, the fermentation product of the strain, the culture medium of the strain, the extract of the strain, the crushed product of the strain, or a mixture thereof, and the composition of the present disclosure may have the anti-aging effect by promoting collagen synthesis.

In one exemplary embodiment of the present disclosure, it was confirmed that the lysate and fermentation product of the novel Lactobacillus sakei LGHNH22021 strain of the present disclosure has the excellent effect of promoting collagen synthesis, as compared to a known comparative strain Lactobacillus sakei KCTC3603, indicating that the Lactobacillus sakei LGHNH22021 strain has a remarkable anti-aging effect.

As used herein, the term “skin regeneration” refers to a recovery process of skin tissues from damage caused by external and internal factors. Damage caused by external factors may include ultraviolet rays, external contaminants, wounds, injury, etc., and damage caused by internal factors may include stress, etc. Specifically, skin regeneration may be due to stimulation of keratinocyte growth, but is not limited thereto.

In the present disclosure, the stimulation of keratinocyte growth may be due to the Lactobacillus sakei LGHNH22021 strain, the culture product of the strain, the fermentation product of the strain, the culture medium of the strain, the extract of the strain, the crushed product of the strain, or a mixture thereof, and the composition of the present disclosure may have the skin regeneration effect by stimulating keratinocyte growth.

In one exemplary embodiment of the present disclosure, it was confirmed that the lysate and fermentation product of the novel Lactobacillus sakei LGHNH22021 strain of the present disclosure has the excellent effect of stimulating keratinocyte growth, as compared to the known comparative strain Lactobacillus sakei KCTC3603, indicating that the Lactobacillus sakei LGHNH22021 strain has a remarkable skin regeneration effect.

As used herein, the term “whitening” encompasses a method of increasing brightness of the skin whose brightness has decreased due to an excess of pigments such as melanin, etc., or maintaining brightness of the skin at a certain level, and the skin with increased brightness by the above method, and it may specifically mean skin whitening.

As used herein, the term “skin whitening” refers to not only brightening skin tone by inhibiting the synthesis of melanin pigment, but also improving skin hyperpigmentation such as spots or freckles caused by ultraviolet rays, hormones, or genetic factors.

As used herein, the term “melanin” is one of the pigments commonly found in nature and has a dark brown or black color. Skin color is determined by the amount of melanin, and the more melanin, the darker the skin' color. Melanin is synthesized from tyrosine, which is one of the amino acids, and forms a polyphenol polymer to have a very stable structure. Because of these structural characteristics, it has resistance to heat and chemicals, antioxidant ability, and resistance to radiation such as ultraviolet rays.

In the present disclosure, the inhibition of melanin synthesis may be due to the Lactobacillus sakei LGHNH22021 strain, the culture product of the strain, the fermentation product of the strain, the culture medium of the strain, the extract of the strain, the crushed product of the strain, or a mixture thereof, and the composition of the present disclosure may have the whitening effect by inhibiting melanin synthesis.

In one exemplary embodiment of the present disclosure, it was confirmed that the lysate and fermentation product of the novel Lactobacillus sakei LGHNH22021 strain of the present disclosure has the excellent effect of inhibiting melanin synthesis, as compared to the known comparative strain Lactobacillus sakei KCTC3603, indicating that the Lactobacillus sakei LGHNH22021 strain has a remarkable skin whitening effect.

As used herein, the term “skin moisturization” refers to all actions that supply moisture to the skin or block evaporation of moisture to maintain the skin's flexibility and to maintain a smooth surface by inducing uniform exfoliation of the skin. “Dry” refers to a state of insufficient skin moisturization. The skin moisturizing effect may help improve skin wrinkles and increase elasticity. Specifically, the skin moisturization may be due to increased production of hyaluronic acid and/or increased expression of aquaporin 3 (AQP3), but is not limited thereto.

As used herein, the term “hyaluronic acid”, which is a type of glycosaminoglycans, refers to a chain-shaped polymer polysaccharide material in which glucuronic acid and N-acetylglucosamine residues are repeatedly linked. Specifically, hyaluronic acid is synthesized by hyaluronan synthase 2 (HAS2), which is a hyaluronic acid synthase enzyme. Therefore, when HAS2 expression is increased, hyaluronic acid synthesis may be promoted.

As used herein, the term “aquaporin 3 (AQP3)” is an integral membrane protein that induces passive transport of water molecules by forming channels in the cell membrane, and selectively allows only water molecules to pass while restricting the movement of other substances.

In the present disclosure, the increased production of hyaluronic acid and/or increased expression of aquaporin 3 may be due to the Lactobacillus sakei LGHNH22021 strain, the culture product of the strain, the fermentation product of the strain, the culture medium of the strain, the extract of the strain, the crushed product of the strain, or a mixture thereof, and the composition of the present disclosure may have the skin moisturizing effect by increasing hyaluronic acid production and/or aquaporin 3 expression.

In one exemplary embodiment of the present disclosure, it was confirmed that the lysate and fermentation product of the novel Lactobacillus sakei LGHNH22021 strain of the present disclosure has the excellent effect of increasing hyaluronic acid production and/or aquaporin 3 expression, as compared to the known comparative strain Lactobacillus sakei KCTC3603, indicating that the Lactobacillus sakei LGHNH22021 strain has a remarkable skin moisturizing effect.

As used herein, the term “skin soothing” refers to alleviating and soothing redness or irritated skin areas, etc., and may be a concept that includes alleviating skin irritation. For example, it may include reducing moisture loss in the skin and/or reducing redness, but is not limited thereto.

As used herein, the term “inflammation” is a type of in vivo response to damage or infection in a specific tissue, wherein the main mediator is immune cells. The purpose of this inflammation is to minimize tissue damage, to eliminate infectious agents, and to regenerate tissues. Specifically, skin soothing and anti-inflammation may be due to inhibition of NO production, but is not limited thereto.

As used herein, the term “NO (nitric oxide)” plays a variety of roles, including eliminating bacteria and tumors, regulating blood pressure, and mediating nerve transmission, etc. However, when an inflammatory response occurs, expression of inducible nitric oxide synthase (iNOS) is increased in related cells, and thus a large amount of NO is produced, and excessively produced NO causes tissue damage, genetic mutation, and nerve damage, and increases vascular permeability, thereby promoting inflammation such as edema, etc. NO production is increased in inflammation-induced cells whereas NO production is suppressed when the skin is soothed, and thus there is an essential correlation therebetween.

In the present disclosure, suppression of NO production may be due to the Lactobacillus sakei LGHNH22021 strain, the culture product of the strain, the fermentation product of the strain, the culture medium of the strain, the extract of the strain, the crushed product of the strain, or a mixture thereof, and the composition of the present disclosure may have the skin soothing and inflammation improving effects by suppressing NO production.

In one exemplary embodiment of the present disclosure, it was confirmed that the lysate and fermentation product of the novel Lactobacillus sakei LGHNH22021 strain of the present disclosure has the excellent effect of suppressing production of nitric oxide (NO) which is a representative redness- and inflammation-causing substance and induces skin irritation, as compared to the known comparative strain Lactobacillus sakei KCTC3603, indicating that the Lactobacillus sakei LGHNH22021 strain has remarkable skin soothing and inflammation improving effects.

As used herein, the term “microbiome” refers to the entire genomic content of the microorganisms inhabiting the human body or the microorganisms themselves, and is a compound word of microbiota, which is a microorganism that inhabits and coexists in the human body, and genome.

As used herein, the term “microbiome enhancement” may be used interchangeably with microbiome balance maintenance, and refers to maintaining the microbiome balance. The microbiome balance may be maintained by promoting the growth of beneficial bacteria while suppressing the growth of harmful bacteria. When the microbiome becomes unbalanced, the skin barrier becomes weak, making it easier for harmful microorganisms to proliferate. Specifically, microbiome enhancement may be due to increased growth of beneficial bacteria and/or inhibition of harmful bacteria growth, but is not limited thereto.

As used herein, the term “beneficial skin bacteria” refers to a strain that helps maintain skin homeostasis by enhancing skin defense by enhancing the skin regeneration effect and promoting skin regeneration by destroying (killing) the cell membrane and inhibiting the growth of harmful bacteria. For example, the beneficial bacteria may include Staphylococcus epidermidis, Staphylococcus warneri, Streptococcus mitis, Micrococcus luteus, Acinetobacter johnsonii, etc., and specifically Staphylococcus epidermidis, but are not limited thereto. More specifically, the beneficial bacteria may be Staphylococcus epidermidis, but are not limited thereto.

The Staphylococcus epidermidis enhances skin defense by inducing the expression of an antimicrobial peptide (AMP) from skin epidermal cells and enhancing the skin regeneration effect; when treated with phenol soluble modulin (PSM), which is a component secreted by Staphylococcus epidermidis, it enhances skin regeneration by destroying cell membranes (death) and inhibiting the growth of harmful bacteria; butyric acid, which is a component secreted by Staphylococcus epidermidis, suppresses the expression of inflammatory factors induced by ultraviolet rays, showing a skin soothing effect; when applying live bacteria to the skin, it has the effect of increasing skin moisture and suppressing moisture loss; when applying live bacteria to the skin, the pH of the skin becomes slightly acidic and the pH environment may be improved, and therefore, substances having the effect of promoting the beneficial bacterium Staphylococcus epidermidis have a beneficial effect on the skin.

As used herein, the term “promoting the proliferation of beneficial skin bacteria” means promoting or improving the growth of the above-described beneficial bacteria. With respect to the objects of the present disclosure, the promotion of the proliferation of beneficial bacteria may be the promotion of the proliferation of Staphylococcus epidermidis, but is not limited thereto.

As used herein, the term “harmful skin bacteria” refers to bacteria that cause inflammation or acne on the skin. The harmful bacteria may be, for example, Staphylococcus aureus, but are not limited thereto.

As used herein, the term “inhibiting the proliferation of harmful skin bacteria” refers to inhibiting the growth of the above-described harmful bacteria. With respect to the objects of the present disclosure, the inhibition of the proliferation of harmful bacteria may be the inhibition of the proliferation of Staphylococcus aureus, but is not limited thereto.

In the present disclosure, the promotion of the proliferation of beneficial skin bacteria and/or the inhibition of the proliferation of harmful skin bacteria may be due to the Lactobacillus sakei LGHNH22021 strain, the culture product of the strain, the fermentation product of the strain, the culture medium of the strain, the extract of the strain, the crushed product of the strain, or a mixture thereof, and the composition of the present disclosure may have the effect of improving the microbiome by promoting the proliferation of beneficial skin bacteria and/or inhibiting the proliferation of harmful skin bacteria.

In one exemplary embodiment of the present disclosure, it was confirmed that the Lactobacillus sakei LGHNH22021 strain promotes the proliferation of the beneficial bacterium Staphylococcus epidermidis and inhibits the growth of the harmful skin bacterium Staphylococcus aureus, indicating that the Lactobacillus sakei LGHNH22021 strain has the microbiome-improving effect.

As used herein, the term “cosmetic composition” may be a formulation selected from the group consisting of a solution, a preparation for external use, a cream, a foam, a nourishing lotion, a softening lotion, a perfume, a pack, softening water, an emulsion, a makeup base, an essence, a soap, a liquid cleaner, a bath product, sunscreen cream, sun oil, a suspension, an emulsion, a paste, a gel, a lotion, powder, a soap, a surfactant-containing cleanser, an oil, a powder foundation, an emulsion foundation, a wax foundation, a patch, and a spray, but is not limited thereto.

As used herein, the term “preparation for external use” refers to a preparation provided for external use, including a skin preparation for external use, a powder preparation for external use, a tablet for external use, a liquid preparation for external use, an ointment for external use, a plaster for external use, and a suppository for external use. The preparation for external use refers to those spread on erosion and ulceration of the skin mucous membrane, such as zinc oxide starch powder, etc., the tablet for external use refers to a solution tablet or a vaginal tablet, and the liquid preparation for external use refers to a liquid preparation containing water, ethanol, oil, etc. as a solvent, and is used for gargling, fomentation, washing, eye drops, nasal drops, etc. Further, the ointment is a semi-solid preparation with appropriate consistency, which is applied to the skin, the plaster is a preparation applied to the skin, which is solid at room temperature and softens at body temperature, and the suppository is a preparation applied to the anus, vagina, and urethra.

The “skin preparation for external use” refers to a preparation that externally acts on the skin among preparations for external use. In the present disclosure, it refers to a skin preparation for external use, containing the erythorbic acid compound or an acceptable salt thereof as an active ingredient. Specifically, the skin preparation for external use may be prepared and used as pharmaceuticals or quasi-drugs in the form of a cream, a gel, a patch, a spray, an ointment, a plaster, a lotion, a liniment preparation, a paste preparation, or a cataplasma preparation, but is not limited thereto.

The cosmetic composition of the present disclosure may further include one or more cosmetically acceptable carriers which are blended in general skin cosmetics, and as common ingredients, for example, oil, water, surfactants, moisturizers, lower alcohols, thickeners, chelating agents, pigments, preservatives, flavoring agents, etc. may be appropriately blended, but are not limited thereto.

The cosmetically acceptable carrier included in the cosmetic composition of the present disclosure may vary according to the formulation of the cosmetic composition.

When the cosmetic formulation of the present disclosure is an ointment, a paste, a cream, or a gel, animal oil, plant oil, wax, paraffin, starch, tracanth, cellulose derivatives, polyethylene glycol, silicone, bentonite, silica, talc, zinc oxide, etc. may be used as the carrier ingredient, but are not limited thereto. These compounds may be used alone or in combination of two or more thereof.

When the formulation of the present disclosure is a solution or an emulsion, a solvent, a solubilizing agent, or an emulsifying agent is used as the carrier component, and for example, water, ethanol, isopropanol, ethyl carbonate, ethyl acetate, benzyl alcohol, benzyl benzoate, propylene glycol, 1,3-butylglycol oil, etc. may be used, and particularly, cottonseed oil, peanut oil, corn germ oil, olive oil, castor oil and sesame oil, glycerol aliphatic esters, polyethylene glycol, or fatty acid esters of sorbitan may be used as the carrier ingredient, but are not limited thereto. These compounds may be used alone or in combination of two or more thereof.

When the formulation of the present disclosure is a suspension, a liquid diluent such as water, ethanol or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, tracanth, etc. may be used as the carrier ingredient, but are not limited thereto. These compounds may be used alone or in combination of two or more thereof.

When the formulation of the present disclosure is a soap, alkali metal salts of fatty acids, fatty acid hemiester salts, fatty acid protein hydrolyzate, isethionate, lanolin derivatives, aliphatic alcohols, plant oils, glycerol, sugar, etc. may be used as the carrier ingredient, but are not limited thereto. These compounds may be used alone or in combination of two or more thereof.

When the formulation of the present disclosure is a powder or a spray, lactose, talc, silica, aluminum hydroxide, calcium silicate, polyamide powder, or a mixture thereof may be used as the carrier ingredient, and specifically, when the formulation is a spray, a propellant such as s chlorofluorohydrocarbon, propane/butane, or dimethyl ether may be included.

On the other hand, all ingredients described in the present disclosure are preferably included in the composition of the present disclosure within the range of not exceeding the maximum usage defined in the Regulation on Safety Standards, etc., for Cosmetics, and China's ‘Safety and Technical Standards for Cosmetics’

In the cosmetic composition of the present disclosure, the Lactobacillus sakei LGHNH22021 strain, the culture product of the strain, the fermentation product of the strain, the culture medium of the strain, the extract of the strain, the crushed product of the strain, or a combination thereof may be included in an amount of 0.000001% to 100%, 0.000001% to 90%, 0.000001% to 80%, 0.000001% to 70%, 0.000001% to 60%, 0.000001% to 50%, 0.000001% to 40%, 0.000001% to 30%, 0.000001% to 20%, 0.000001% to 15%, 0.000001% to 10%, 0.00001% to 90%, 0.00001% to 80%, 0.00001% to 70%, 0.00001% to 60%, 0.00001% to 50%, 0.00001% to 40%, 0.00001% to 30%, 0.00001% to 20%, 0.00001% to 15%, 0.00001% to 10%, 0.0001% to 90%, 0.0001% to 80%, 0.0001% to 70%, 0.0001% to 60%, 0.0001% to 50%, 0.0001% to 40%, 0.0001% to 30%, 0.0001% to 20%, 0.0001% to 15%, 0.0001% to 10%, 0.001% to 90%, 0.001% to 80%, 0.001% to 70%, 0.001% to 60%, 0.001% to 50%, 0.001% to 40%, 0.001% to 30%, 0.001% to 20%, 0.001% to 15%, 0.001% to 10%, 0.01% to 90%, 0.01% to 80%, 0.01% to 70%, 0.01% to 60%, 0.01% to 50%, 0.01% to 40%, 0.01% to 30%, 0.01% to 20%, 0.01% to 15%, 0.01% to 10%, 0.1% to 90%, 0.1% to 80%, 0.1% to 70%, 0.1% to 60%, 0.1% to 50%, 0.1% to 40%, 0.1% to 30%, 0.1% to 20%, 0.1% to 15%, 0.1% to 10%, 1% to 90%, 1% to 80%, 1% to 70%, 1% to 60%, 1% to 50%, 1% to 40%, 1% to 30%, 1% to 20%, 1% to 15%, or 1% to 10%, based on the total weight of the cosmetic composition, but is not limited thereto, for example, may be included in an amount of 0.001% to 100%, specifically, 0.01% to 30%, but is not limited thereto.

Still another aspect of the present disclosure provides a quasi-drug composition for skin improvement, the quasi-drug composition including any one or more of the Lactobacillus sakei LGHNH22021 strain, a culture product of the strain, a fermentation product of the strain, a culture medium of the strain, an extract of the strain, and a crushed product of the strain.

The “Lactobacillus sakei LGHNH22021”, “strain”, “culture product”, “culture medium”, “crushed product”, “fermentation product”, “extract” and “skin improvement”, etc. are as described above.

Specifically, the quasi-drug composition may be for skin improvement, wherein the skin improvement may be one or more selected from the group consisting of anti-aging, wrinkle improvement, elasticity enhancement, skin regeneration, skin whitening, skin moisturization, skin soothing, anti-inflammation, and skin microbiome enhancement, but is not limited thereto.

As used herein, the term “quasi-drug” may be selected from the group consisting of body cleansers, disinfectant cleaners, cleaners, kitchen cleaners, cleaning agents, toothpaste, mouthwash, wet tissue, detergents, soap, hand wash, hair cleaners, hair softeners, humidifier fillers, masks, ointments, and filter fillers, but is not limited thereto.

In addition to the above ingredients, the quasi-drug composition of the present disclosure may further include a pharmaceutically acceptable carrier, excipient, or diluent, as needed. The pharmaceutically acceptable carrier, excipient, or diluent is not limited, as long as it does not impair the effects of the present disclosure, and may include, for example, fillers, extenders, binders, wetting agents, disintegrants, surfactants, lubricants, sweeteners, fragrances, preservatives, etc.

Representative examples of the pharmaceutically acceptable carrier, excipient, or diluent of the present disclosure may include lactose, dextrose, sucrose, sorbitol, mannitol, xylitol, maltitol, starch, gelatin, glycerin, acacia rubber, alginate, calcium phosphate, calcium carbonate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, propylene glycol, polyethylene glycol, vegetable oil, injectable esters, Witepsol, Macrogol, Tween 61, cacao butter, laurin butter, etc.

When the strain of the present disclosure or the fermentation product thereof is used as a quasi-drug, it may further include one or more active ingredients that exhibit the same or similar functions, and may include, for example, the known ingredients for promotion of beneficial skin bacteria, inhibition of harmful skin bacteria, skin soothing, skin inflammation improvement, skin whitening, skin regeneration, wound healing, etc. When additional ingredients for wrinkle improvement, skin whitening, skin trouble improvement, and skin moisturizing are included, the antioxidant, whitening, anti-aging, skin elasticity improvement, microbiome improvement, skin moisturizing, skin soothing, inflammation improvement, and skin barrier enhancement effects of the composition of the present disclosure may be further improved. When the above ingredients are added, skin safety due to combined use, ease of formulation, and stability of the active ingredients may be taken into consideration.

The quasi-drug composition of the present disclosure may further include one or two or more components selected from the group consisting of retinoic acid, TGF, animal placenta-derived proteins, betulinic acid, and a chlorella extract as a skin anti-aging component known in the art; non-steroidal flufenamic acid, ibuprofen, benzydamine, indomethacin, prednisolone, dexamethasone, allantoin, azene, hydrocortisone as an anti-inflammatory components known in the art; derivatives thereof and various plant extracts. Additional components may be included in an amount of 0.0001% by weight to 10% by weight with respect to the total weight of the composition, and the above range of content may be adjusted according to requirements such as skin safety, ease of formulation of the strain or the fermentation product of the present invention, etc.

The formulation method, dosage, usage method, components, etc. of the quasi-drug may be appropriately selected from common techniques known in the art.

In the quasi-drug composition of the present disclosure, the Lactobacillus sakei LGHNH22021 strain, the culture product of the strain, the fermentation product of the strain, the culture medium of the strain, the extract of the strain, the crushed product of the strain, or a combination thereof may be included in an amount of 0.000001% to 100%, 0.000001% to 90%, 0.000001% to 80%, 0.000001% to 70%, 0.000001% to 60%, 0.000001% to 50%, 0.000001% to 40%, 0.000001% to 30%, 0.000001% to 20%, 0.000001% to 15%, 0.000001% to 10%, 0.00001% to 90%, 0.00001% to 80%, 0.00001% to 70%, 0.00001% to 60%, 0.00001% to 50%, 0.00001% to 40%, 0.00001% to 30%, 0.00001% to 20%, 0.00001% to 15%, 0.00001% to 10%, 0.0001% to 90%, 0.0001% to 80%, 0.0001% to 70%, 0.0001% to 60%, 0.0001% to 50%, 0.0001% to 40%, 0.0001% to 30%, 0.0001% to 20%, 0.0001% to 15%, 0.0001% to 10%, 0.001% to 90%, 0.001% to 80%, 0.001% to 70%, 0.001% to 60%, 0.001% to 50%, 0.001% to 40%, 0.001% to 30%, 0.001% to 20%, 0.001% to 15%, 0.001% to 10%, 0.01% to 90%, 0.01% to 80%, 0.01% to 70%, 0.01% to 60%, 0.01% to 50%, 0.01% to 40%, 0.01% to 30%, 0.01% to 20%, 0.01% to 15%, 0.01% to 10%, 0.1% to 90%, 0.1% to 80%, 0.1% to 70%, 0.1% to 60%, 0.1% to 50%, 0.1% to 40%, 0.1% to 30%, 0.1% to 20%, 0.1% to 15%, 0.1% to 10%, 1% to 90%, 1% to 80%, 1% to 70%, 1% to 60%, 1% to 50%, 1% to 40%, 1% to 30%, 1% to 20%, 1% to 15%, or 1% to 10%, based on the total weight of the quasi-drug composition, but is not limited thereto, for example, in an amount of 0.001% to 100%, specifically, 0.01% to 30%, but is not limited thereto.

Still another aspect of the present disclosure provides a food composition for skin improvement, the food composition including any one or more of the Lactobacillus sakei LGHNH22021 strain, a culture product of the strain, a fermentation product of the strain, a culture medium of the strain, an extract of the strain, and a crushed product of the strain.

The “Lactobacillus sakei LGHNH22021”, “strain”, “culture product”, “culture medium”, “crushed product”, “fermentation product”, “extract” and “skin improvement”, etc. are as described above.

Specifically, the food composition may be for skin improvement, wherein the skin improvement may be one or more selected from the group consisting of anti-aging, wrinkle improvement, elasticity enhancement, skin regeneration, skin whitening, skin moisturization, skin soothing, anti-inflammation, and skin microbiome enhancement, but is not limited thereto.

As used herein, the term “food” may include all foods in the ordinary sense, such as meat, sausage, bread, chocolate, candy, snacks, confectionery, pizza, ramen, other noodles, gum, and dairy products including ice cream, various soups, beverages, teas, drinks, alcoholic beverages, vitamin complexes, health functional foods, etc., and is not limited, as long as it may include the Lactobacillus sakei LGHNH22021 strain of the present disclosure, a culture product of the strain, a fermentation product of the strain, a culture medium of the strain, an extract of the strain, and a crushed product of the strain. Further, the food may include juices, teas, jellies, and juices which are prepared by adding the strain according to the present disclosure, the culture product, fermentation product, culture medium, extract, and crushed product of the strain as a main ingredient, and may include forms such as pills, powders, granules, tablets, capsules, liquids, etc.

The health functional food (functional food) is the same term as food for special health use (FoSHU), and refers to a food having high medicinal and medical effects, which is processed to effectively exert a body-regulating function as well as to supply nutrients. Here, the term “functional” means that it is taken for the purpose of controlling nutrients with respect to structures and functions of the human body or of obtaining effects beneficial for health care, such as physiological effects. The food of the present disclosure may be prepared by a method commonly used in the art, and may be prepared by adding raw materials and ingredients which are commonly added in the art during the preparation. In addition, the formulation of the food may be prepared without limitation, as long as it is acceptable as food. The food composition of the present disclosure may be prepared as various types of formulations. Unlike general drugs, the food composition includes a food as a raw material, and therefore, it has advantages of being free from side effects that may occur when taken for a long period of time, and of being highly portable, and therefore, the food of the present disclosure may be ingested as an auxiliary to enhance the immune enhancement effect.

The health food refers to a food which brings about an active health-preserving or enhancing effect, as compared to the common food, and the health supplement food refers to a food for the purpose of health supplementation. In some cases, the terms of health functional food, health food, and health supplement food are used interchangeably.

Specifically, the health functional food is a food prepared by adding any one or more of the Lactobacillus sakei LGHNH22021, a culture product of the strain, a fermentation product of the strain, a culture medium of the strain, an extract of the strain, and a crushed product of the strain to a food material such as beverage, tea, spice, gum, chips, etc., or by encapsulation, powdering, or suspension, and brings about a specific effect to health when consumed. However, unlike general drugs, it has advantages of being free from side effects that may occur when a drug is administered for a long period of time because it is prepared by using food as raw materials.

The composition may further include a physiologically acceptable carrier, and the type of carrier is not particularly limited, and any carrier may be used as long as it is commonly used in the art.

Further, the composition may further include additional ingredients which are commonly used in food compositions to improve odor, taste, vision, etc., and may include, for example, vitamins A, C, D, E, B1, B2, B6, B12, niacin, biotin, folate, panthotenic acid, etc. Furthermore, it may include minerals such as zinc (Zn), iron (Fe), calcium (Ca), chromium (Cr), magnesium (Mg), manganese (Mn), copper (Cu), chromium (Cr), etc. In addition, it may include amino acids such as lysine, tryptophan, cysteine, valine, etc.

Moreover, the composition may include food additives such as preservatives (potassium sorbate, sodium benzoate, salicylic acid, sodium dehydroacetate, etc.), disinfectants (bleaching powder and high-grade bleaching powder, sodium hypochlorite, etc.), antioxidants (butylhydroxyanisole (BHA), butylhydroxytoluene (BHT), etc.), coloring agents (tar color, etc.), color former agents (sodium nitrite, etc.), bleaching agents (sodium sulfite, etc.), seasoning (sodium glutamate (MSG), etc.), sweeteners (dulcin, cyclamate, saccharin, sodium, etc.), flavoring agents (vanillin, lactones, etc.), swelling agents (alum, potassium hydrogen D-tartrate), reinforcing agents, emulsifying agents, thickener (thickening agents), coating agents, gum bases, foam inhibitors, solvents, enhancers, etc. The additives may be selected and used in an appropriate amount according to the type of food.

Any one or more of the Lactobacillus sakei LGHNH22021, a culture product of the strain, a fermentation product of the strain, a culture medium of the strain, an extract of the strain, and a crushed product of the strain may be added as it is or used together with other food or food ingredients, and may be appropriately used according to a common method. The mixing ratio of the active ingredients may be appropriately determined according to the purpose thereof (prevention, health, or therapeutic treatment). In general, during the production of food or beverage, the food composition of the present disclosure may be added in an amount of 50 parts by weight or less, specifically 20 parts by weight or less with respect to the food or beverage. However, when it is intended for long-term intake for health care and hygiene, the composition may be used in an amount less than the above range, and since it does not have any safety risk, the active ingredient may also be used in an amount exceeding the above range.

As an example of the food composition of the present disclosure, it may be used as a health beverage composition, and in this case, various flavoring agents or natural carbohydrates may be added as additional ingredients as in the case of common beverages. The above-mentioned natural carbohydrates may include monosaccharides such as glucose and fructose; disaccharides such as maltose and sucrose; polysaccharides such as dextrin and cyclodextrin; and sugar alcohols such as xylitol, sorbitol, erythritol, etc. Sweeteners may include natural sweeteners such as thaumatin and stevia extract; and synthetic sweeteners such as saccharin, aspartame, etc. A proportion of the natural carbohydrate is generally about 0.01 g to 0.04 g, specifically about 0.02 g to 0.03 g per 100 mL of the composition of the present disclosure.

In addition to the above, the health beverage composition may include various nutritional supplements, vitamins, electrolytes, flavoring agents, coloring agents, pectic acid and salts thereof, alginic acid and salts thereof, organic acids, protective colloid thickening agents, pH adjusters, stabilizers, preservatives, glycerin, alcohol, or carbonating agents, etc. In addition, it may include flesh for the production of natural fruit juices, fruit juice drinks or vegetable drinks. These ingredients may be used independently or in a mixture. The proportion of such additives is not critical, but is generally selected in the range of 0.01 part by weight to 0.1 part by weight, based on 100 parts by weight of the composition of the present disclosure.

In the food composition of the present disclosure, the Lactobacillus sakei LGHNH22021 strain, the culture product of the strain, the fermentation product of the strain, the culture medium of the strain, the extract of the strain, the crushed product of the strain, or a combination thereof may be included in an amount of 0.000001% to 100%, 0.000001% to 90%, 0.000001% to 80%, 0.000001% to 70%, 0.000001% to 60%, 0.000001% to 50%, 0.000001% to 40%, 0.000001% to 30%, 0.000001% to 20%, 0.000001% to 15%, 0.000001% to 10%, 0.00001% to 90%, 0.00001% to 80%, 0.00001% to 70%, 0.00001% to 60%, 0.00001% to 50%, 0.00001% to 40%, 0.00001% to 30%, 0.00001% to 20%, 0.00001% to 15%, 0.00001% to 10%, 0.0001% to 90%, 0.0001% to 80%, 0.0001% to 70%, 0.0001% to 60%, 0.0001% to 50%, 0.0001% to 40%, 0.0001% to 30%, 0.0001% to 20%, 0.0001% to 15%, 0.0001% to 10%, 0.001% to 90%, 0.001% to 80%, 0.001% to 70%, 0.001% to 60%, 0.001% to 50%, 0.001% to 40%, 0.001% to 30%, 0.001% to 20%, 0.001% to 15%, 0.001% to 10%, 0.01% to 90%, 0.01% to 80%, 0.01% to 70%, 0.01% to 60%, 0.01% to 50%, 0.01% to 40%, 0.01% to 30%, 0.01% to 20%, 0.01% to 15%, 0.01% to 10%, 0.1% to 90%, 0.1% to 80%, 0.1% to 70%, 0.1% to 60%, 0.1% to 50%, 0.1% to 40%, 0.1% to 30%, 0.1% to 20%, 0.1% to 15%, 0.1% to 10%, 1% to 90%, 1% to 80%, 1% to 70%, 1% to 60%, 1% to 50%, 1% to 40%, 1% to 30%, 1% to 20%, 1% to 15%, or 1% to 10%, based on the total weight of the food composition, but is not limited thereto, and for example, in an amount of 0.001% to 100%, specifically 0.01% to 30%, but is not limited thereto.

Still another aspect of the present disclosure provides a composition for promoting the proliferation of Staphylococcus epidermidis which is a beneficial skin bacterium, the composition including any one or more of the Lactobacillus sakei LGHNH22021 strain, a culture product of the strain, a fermentation product of the strain, a culture medium of the strain, an extract of the strain, and a crushed product of the strain.

The composition may be a cosmetic composition, quasi-drug composition, or food composition, but is not limited thereto.

The “Lactobacillus sakei LGHNH22021”, “beneficial skin bacterium” and “promoting the proliferation of beneficial skin bacterium”, etc. are as described above.

Still another aspect of the present disclosure provides a composition for inhibiting the proliferation of Staphylococcus aureus which is a harmful skin bacterium, the composition including any one or more of the Lactobacillus sakei LGHNH22021 strain, a culture product of the strain, a fermentation product of the strain, a culture medium of the strain, an extract of the strain, and a crushed product of the strain.

The composition may be a cosmetic composition, quasi-drug composition, or food composition, but is not limited thereto.

The “Lactobacillus sakei LGHNH22021”, “harmful skin bacterium” and “inhibiting the proliferation of harmful skin bacterium”, etc. are as described above.

Still another aspect of the present disclosure provides a method for preparing a fermentation product, the method including fermenting the Lactobacillus sakei LGHNH22021 strain.

The method for preparing a fermentation product of the present disclosure may be appropriately selected from common techniques known in the art.

The “Lactobacillus sakei LGHNH22021”, “strain”, “fermentation” and “fermentation product”, etc. are as described above.

Still another aspect of the present disclosure provides a method for preparing a lysate, the method including crushing the Lactobacillus sakei LGHNH22021 strain.

The method for preparing a lysate of the present disclosure may be appropriately selected from common techniques known in the art.

The “Lactobacillus sakei LGHNH22021”, “strain”, “fermentation” and “lysate”, etc. are as described above.

Still another aspect of the present disclosure provides a method for improving skin conditions, the method including applying to, a subject' skin, a composition including any one or more of the Lactobacillus sakei LGHNH22021 strain, a culture product of the strain, a fermentation product of the strain, a culture medium of the strain, an extract of the strain, and a crushed product of the strain.

The skin improvement may be any one or more selected from the group consisting of anti-aging, wrinkle improvement, elasticity enhancement, skin regeneration, skin whitening, skin moisturization, skin soothing, anti-inflammation, and skin microbiome enhancement, but is not limited thereto.

The composition may be a cosmetic composition, quasi-drug composition, or food composition, but is not limited thereto.

In the present disclosure, the “subject” refers to all animals, including human being, who need or are likely to need one or more of the treatments selected from the group consisting of anti-aging, wrinkle improvement, elasticity improvement, skin regeneration, skin whitening, skin moisturizing, skin soothing, anti-inflammation, and skin microbiome improvement. The animals may be humans as well as mammals such as cows, horses, sheep, pigs, goats, camels, antelopes, dogs, and cats, which require treatment for similar symptoms, but are not limited thereto.

The “Lactobacillus sakei LGHNH22021”, “strain”, “culture product”, “culture medium”, “crushed product”, “fermentation product”, “extract”, “skin improvement”, “cosmetic composition”, “quasi-drug composition”, and “food composition”, etc. are as described above.

Still another aspect of the present disclosure provides use of a composition including any one or more of the Lactobacillus sakei LGHNH22021 strain, a culture product of the strain, a fermentation product of the strain, a culture medium of the strain, an extract of the strain, and a crushed product of the strain in improving skin conditions.

The composition may be a cosmetic composition, quasi-drug composition, or food composition, but is not limited thereto.

The skin improvement may be any one or more selected from the group consisting of anti-aging, wrinkle improvement, elasticity enhancement, skin regeneration, skin whitening, skin moisturization, skin soothing, anti-inflammation, and skin microbiome enhancement, but is not limited thereto.

The “Lactobacillus sakei LGHNH22021”, “strain”, “culture product”, “culture medium”, “crushed product”, “fermentation product”, “extract”, “skin improvement”, “cosmetic composition”, “quasi-drug composition” and “food composition”, etc. are as described above.

Still another aspect of the present disclosure provides use of Lactobacillus sakei LGHNH22021 strain deposited with Accession No. KCTC 15087BP in improving the skin.

The skin improvement may be any one or more selected from the group consisting of anti-aging, wrinkle improvement, elasticity enhancement, skin regeneration, skin whitening, skin moisturization, skin soothing, anti-inflammation, and skin microbiome enhancement, but is not limited thereto.

### Advantageous Effects

Since a Lactobacillus sakei LGHNH22021 strain of the present disclosure, a culture product of the strain, a fermentation product of the strain, a culture medium of the strain, an extract of the strain, or a crushed product of the strain has excellent skin improvement effects, it has high applicability as a cosmetic composition, a quasi-drug composition, and a food composition for skin improvement. Accordingly, various industrial applications, such as in cosmetics, quasi-drugs, and foods, etc., may be expected.

### BRIEF DESCRIPTION OF THE DRAWING

FIG. 1 shows results of analyzing the effects of a lysate or fermentation product of a Lactobacillus sakei LGHNH22021 strain of the present disclosure on promoting collagen synthesis.

### DETAILED DESCRIPTION OF PREFERRED EMBODIMENTS

Hereinafter, the present disclosure will be described in more detail with reference to exemplary embodiments. However, these exemplary embodiments are only for illustrating the present disclosure, and the scope of the present disclosure is not intended to be limited by these exemplary embodiments.

### Example 1. Isolation of Novel Lactobacillus sakei LGHNH22021 Strain from Achillea millefolium

A novel Lactobacillus sakei strain was isolated from Achillea millefolium.

In detail, Achillea millefolium leaves were washed several times with autoclaved water. Subsequently, 5 g of leaves were cut into small pieces, placed in 50 ml of PBS, and became homogeneous by vortexing. Subsequently, it was diluted with PBS and dispensed into an MRS solid medium (BD, 288130) in an amount of 100 μl per dilution, plated on a flat surface, and the plated solid medium was cultured at 30° C. for 2 days.

The cultured strain was isolated and identified, and confirmed to be Lactobacillus sakei (L. sakei), and named Lactobacillus sakei LGHNH22021, which was deposited under the Budapest Treaty in the international depository authority, Korean Culture Center of Microorganisms (KCTC), on Sep. 20, 2022, with the Accession No. KCTC 15087BP.

A base sequence of 16s rRNA of the strain is shown in SEQ ID NO: 1.

16s rRNA sequence of Lactobacillus sakei
LGHNH22021 strain (SEQ ID NO: 1):
GCGTGCCTAATACATGCAAGTCGAACGCACTCTCGTTTAGATTGAAG
GAGCTTGCTCCTGATTGATAAACATTTGAGTGAGTGGCGGACGGGTGAG
TAACACGTGGGTAACCTGCCCTAAAGTGGGGGATAACATTTGGAAACAG
ATGCTAATACCGCATAAAACCTAACACCGCATGGTGTAGGGTTGAAAGA
TGGTTTCGGCTATCACTTTAGGATGGACCCGCGGTGCATTAGTTAGTTG
GTGAGGTAAAGGCTCACCAAGACCGTGATGCATAGCCGACCTGAGAGGG
TAATCGGCCACACTGGGACTGAGACACGGCCCAGACTCCTACGGGAGGC
AGCAGTAGGGAATCTTCCACAATGGACGAAAGTCTGATGGAGCAACGCC
GCGTGAGTGAAGAAGGTTTTCGGATCGTAAAACTCTGTTGTTGGAGAAG
AATGTATCTGATAGTAACTGATCAGGTAGTGACGGTATCCAACCAGAAA
GCCACGGCTAACTACGTGCCAGCAGCCGCGGTAATACGTAGGTGGCAAG
CGTTGTCCGGATTTATTGGGCGTAAAGCGAGCGCAGGCGGTTTCTTAAG
TCTGATGTGAAAGCCTTCGGCTCAACCGAAGAAGTGCATCGGAAACTGG
GAAACTTGAGTGCAGAAGAGGACAGTGGAACTCCATGTGTAGCGGTGAA
ATGCGTAGATATATGGAAGAACACCAGTGGCGAAGGCGGCTGTCTGGTC
TGTAACTGACGCTGAGGCTCGAAAGCATGGGTAGCAAACAGGATTAGAT
ACCCTGGTAGTCCATGCCGTAAACGATGAGTGCTAGGTGTTGGAGGGTT
TCCGCCCTTCAGTGCCGCAGCTAACGCATTAAGCACTCCGCCTGGGGAG
TACGACCGCAAGGTTGAAACTCAAAGGAATTGACGGGGGCCCGCACAAG
CGGTGGAGCATGTGGTTTAATTCGAAGCAACGCGAAGAACCTTACCAGG
TCTTGACATCCTTTGACCACTCTAGAGATAGAGCTTTCCCTTCGGGGAC
AAAGTGACAGGTGGTGCATGGTTGTCGTCAGCTCGTGTCGTGAGATGTT
GGGTTAAGTCCCGCAACGAGCGCAACCCTTATTACTAGTTGCCAGCATT
TAGTTGGGCACTCTAGTGAGACTGCCGGTGACAAACCGGAGGAAGGTGG
GGACGACGTCAAATCATCATGCCCCTTATGACCTGGGCTACACACGTGC
TACAATGGATGGTACAACGAGTTGCGAGACCGCGAGGTTTAGCTAATCT
CTTAAAACCATTCTCAGTTCGGATTGTAGGCTGCAACTCGCCTACATGA
AGCCGGAATCGCTAGTAATCGCGGATCAGCATGCCGCGGTGAATAC

### Example 2. Preparation of Fermentation Product and Lysate of Novel Lactobacillus sakei LGHNH22021 Strain

### Example 2-1. Preparation of Fermentation Product of Novel Lactobacillus sakei LGHNH22021 Strain

Lactobacillus sakei LGHNH22021 strain was inoculated into MRS medium, cultured with shaking at 30° C. and 200 rpm for 4 days, and centrifuged to take a supernatant, thereby preparing a fermentation product of Lactobacillus sakei LGHNH22021 strain.

In the supernatant taken, the culture medium of the LGHNH22021 strain of the present disclosure and a crushed product obtained by crushing the culture product were included. Since the centrifuged supernatant is produced as a result of decomposing organic compounds by the strain, it corresponds to the fermentation product of Lactobacillus sakei LGHNH22021 strain.

### Example 2-2. Preparation of Lysate (Crushed Product) of Novel Lactobacillus sakei LGHNH22021 Strain

The cells obtained after centrifuging the culture product in Example 2-1 were suspended at 5% in purified water and then sonicated (Amplitude 40, Process time 40 min, Pulse-ON time 1 S, Plus-OFF time 2 S), and the cells were crushed and filtered, and finally, a Lactobacillus lysate (crushed product) was prepared.

### Example 2-3. Preparation of Fermentation Product and Lysate of Lactobacillus sakei Reference Strain (KCTC3603)

Lactobacillus sakei reference strain (KCTC3603) was purchased from the Korean Culture Center of Microorganisms (KCTC). A lysate and a fermentation product of the reference strain were prepared in the same manner as the preparation of the lysate and the fermentation product of Lactobacillus sakei LGHNH22021 in Example 2-1 and Example 2-2.

### Example 3. Evaluation of Anti-Aging Effect of Novel Lactobacillus sakei LGHNH22021 Strain

To verify the anti-aging effect of the novel strain, human fibroblast HS68 cells were treated with a negative control (control, DMEM containing 0% serum); the lysate (LS-LG) and the fermentation product (LS-LGF) of Lactobacillus sakei LGHNH22021 strain of the present disclosure; the lysate (LS-TS) and the fermentation product (LS-TSF) of Lactobacillus sakei reference strain (KCTC3603); and a broth at each concentration for 24 hours.

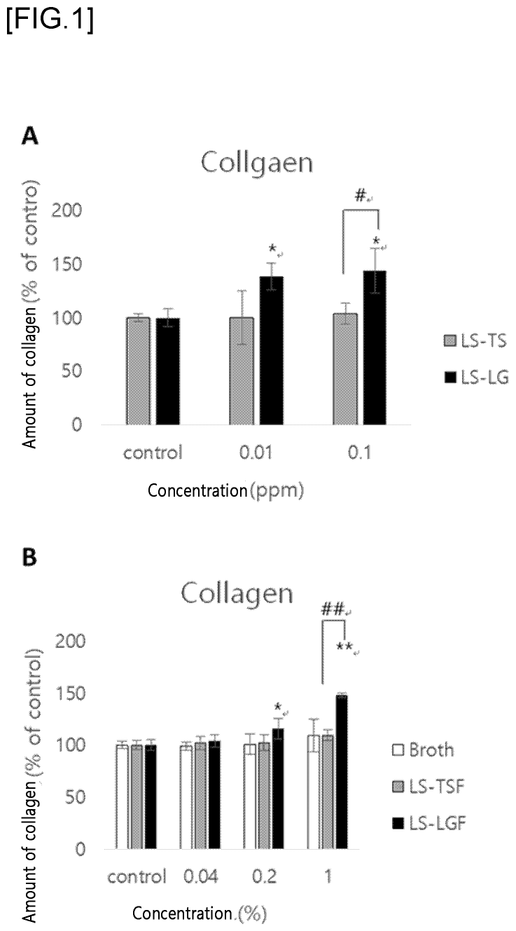

Subsequently, the collagen synthesis rate was measured using a procollagen type IC-peptide (PIP) kit. The collagen synthesis efficacy of the sample was calculated compared to the negative control (control, DMEM containing 0% serum), and the value was determined. The results are shown in FIG. 1 and Tables 1 and 2 below.

TABLE 1

| | Amount of collagen | | | | | | |
|---|---|---|---|---|---|---|---|
| | (% of control) | | | | p-value | | p-value |
| Concentration | Average | | STDEV | | (vs control) | | (LS-LG vs |
| (ppm) | LS-TS | LS-LG | LS-TS | LS-LG | LS-TS | LS-LG | LS-TS) |
| control | 100 | 100 | 3.947205 | 8.647987 | — | — | 0.1070 |
| 0.01 | 100.3285 | 138.1979 | 25.09889 | 12.33891 | 0.7921 | 0.0261 | 0.5068 |
| 0.1 | 104.0916 | 143.7285 | 9.886548 | 20.89053 | 0.2698 | 0.0285 | 0.0190 |

TABLE 2

| | Amount of collagen | | | | | | | | | |
|---|---|---|---|---|---|---|---|---|---|---|
| | (% of control) | | | | | | p-value | | | p-value |
| Concentration | Average | | | STDEV | | | (vs control) | | | (LS-LGF vs |
| (%) | Broth | LS-TSF | LS-LGF | Broth | LS-TSF | LS-LGF | Broth | LS-TSF | LS-LGF | LS-TSF) |
| control | 100 | 100 | 100 | 3.624575 | 4.263715 | 5.147679 | — | — | — | 0.603 |
| 0.04 | 99.31658 | 101.8315 | 104.2121 | 3.947205 | 6.30125 | 5.929361 | 0.1197 | 0.4502 | 0.5068 | 0.981 |
| 0.2 | 100.6526 | 102.3016 | 115.8843 | 9.887034 | 7.31985 | 9.67853 | 0.0997 | 0.2814 | 0.0705 | 0.449 |
| 1 | 109.1588 | 109.4168 | 147.6949 | 15.88655 | 5.014762 | 1.992213 | 0.1072 | 0.1921 | 0.0294 | 0.001 |

As shown in FIG. 1 and Tables 1 and 2, it was confirmed that the lysate (A in FIG. 1, Table 1) and the fermentation product (B in FIG. 1, Table 2) of Lactobacillus sakei LGHNH22021 strain of the present disclosure increased the collagen synthesis in a concentration-dependent manner, and they have the more remarkable collagen synthesis promotion effects than the lysate and the fermentation product of the comparative strain KCTC3603.

Accordingly, it was confirmed that Lactobacillus sakei LGHNH22021 strain of the present disclosure has remarkable anti-aging, wrinkle improvement, and elasticity improvement effects.

### Example 4. Evaluation of Skin Regeneration Effect of Novel Lactobacillus sakei LGHNH22021 Strain

To verify the skin regeneration effect of the novel strain, human keratinocyte HaCaT cells were treated with a negative control (control, DMEM containing 0% serum); the lysate (LS-LG) and the fermentation product (LS-LGF) of Lactobacillus sakei LGHNH22021 strain of the present disclosure; the lysate (LS-TS) and the fermentation product (LS-TSF) of Lactobacillus sakei reference strain (KCTC3603); and a broth at each concentration for 24 hours.

Subsequently, CCK-8 solution was added, and allowed to react for 1 hour and 30 minutes, and absorbance at 460 nm was measured to calculate the proliferation rate of keratinocyte HaCaT cells, compared to the negative control group. The results are shown in FIG. 2 and Tables 3 and 4 below.

TABLE 3

| | Cell proliferation | | | | | | |
|---|---|---|---|---|---|---|---|
| | (% of control) | | | | p-value | | p-value |
| Concentration | average | | STDEV | | (vs control) | | (LS-LG vs |
| (ppm) | LS-TS | LS-LG | LS-TS | LS-LG | LS-TS | LS-LG | LS-TS) |
| control | 100 | 100 | 5.0245 | 7.1465 | — | — | 0.392 |
| 0.01 | 99.0746 | 110.4153 | 13.2598 | 8.54879 | 0.7681 | 0.027 | 0.312 |
| 0.1 | 103.7415 | 135.4132 | 9.31547 | 11.4789 | 0.8572 | 0.028 | 0.033 |

TABLE 4

| | Cell proliferation | | | | | | | | | |
|---|---|---|---|---|---|---|---|---|---|---|
| | (% of control) | | | | | | p-value | | | p-value |
| Concentration | average | | | STDEV | | | (vs control) | | | (LS-LGF vs |
| (%) | Broth | LS-TSF | LS-LGF | Broth | LS-TSF | LS-LGF | Broth | LS-TSF | LS-LGF | LS-TSF) |
| control | 100 | 100 | 100 | 2.1498 | 5.96314 | 3.8426 | — | — | — | 0.593 |
| 0.04 | 100.347 | 99.0135 | 105.1473 | 5.0197 | 10.4713 | 5.7416 | 0.378 | 0.855 | 0.0541 | 0.123 |
| 0.2 | 105.9315 | 102.3016 | 111.7413 | 8.2461 | 15.1798 | 4.4785 | 0.198 | 0.279 | 0.0541 | 0.1266 |
| 1 | 110.1657 | 101.3748 | 132.4178 | 6.1432 | 8.3146 | 6.6874 | 0.212 | 0.180 | 0.045 | 0.0222 |

As shown in FIG. 2 and Tables 3 and 4, it was confirmed that the lysate (A in FIG. 2, Table 3) and the fermentation product (B in FIG. 2, Table 4) of Lactobacillus sakei LGHNH22021 strain of the present disclosure increased the keratinocyte growth in a concentration-dependent manner, and they have more remarkable keratinocyte growth promotion effects than the lysate and the fermentation product of the comparative strain KCTC3603.

Accordingly, it was confirmed that Lactobacillus sakei LGHNH22021 strain of the present disclosure has the remarkable skin regeneration effects.

### Example 5. Evaluation of Skin Whitening Effect of Novel Lactobacillus sakei LGHNH22021 Strain

To verify the skin whitening effect of the novel strain, mouse melanoma cell line B16F1 was treated with a negative control (control, DMEM containing 0% serum); the lysate (LS-LG) and the fermentation product (LS-LGF) of Lactobacillus sakei LGHNH22021 strain of the present disclosure; the lysate (LS-TS) and the fermentation product (LS-TSF) of Lactobacillus sakei reference strain (KCTC3603); and a broth at each concentration for 72 hours.

Subsequently, the cells were collected, centrifuged, and the supernatant was discarded. 100 μL of 1N NaOH/10% DMSO solution was added and dissolved at 80° C. for 20 minutes. Subsequently, absorbance at 400 nm was measured using a microplate reader, and the melanin synthesis amount was calculated using a melanin standard curve and expressed as a ratio with respect to the negative control group. The results are shown in FIG. 3 and Tables 5 and 6 below.

TABLE 5

| | Amount of melanin | | | | | | |
|---|---|---|---|---|---|---|---|
| | (% of control) | | | | p-value | | p-value |
| Concentration | average | | STDEV | | (vs control) | | (LS-LG vs |
| (ppm) | LS-TS | LS-LG | LS-TS | LS-LG | LS-TS | LS-LG | LS-TS) |
| control | 100 | 100 | 9.938183 | 5.407212 | — | — | 0.9232 |
| 0.01 | 102.7952 | 81.76152 | 10.09359 | 12.39761 | 0.103 | 0.03 | 0.5345 |
| 0.1 | 98.5359 | 80.63022 | 4.993107 | 6.183181 | 0.110 | 0.038 | 0.0261 |
| 1 | 99.18015 | 78.53066 | 7.179256 | 8.690604 | 0.095 | 0.009 | 0.0285 |

TABLE 6

| | Amount of melanin | | | | | | | | | |
|---|---|---|---|---|---|---|---|---|---|---|
| | (% of control) | | | | | | p-value | | | p-value |
| Concentration | average | | | STDEV | | | (vs control) | | | (LS-LGF vs |
| (%) | Broth | LS-TSF | LS-LGF | Broth | LS-TSF | LS-LGF | Broth | LS-TSF | LS-LGF | LS-TSF) |
| control | 100 | 100 | 100 | 1.692934 | 3.21365 | 4.98298 | — | — | — | 0.3180 |
| 0.04 | 105.4048 | 104.3875 | 95.38647 | 2.259545 | 5.61204 | 2.934333 | 0.2368 | 0.7411 | 0.3347 | 0.1144 |
| 0.2 | 111.3264 | 100.5934 | 83.09492 | 6.023772 | 3.6572 | 5.122205 | 0.6548 | 0.6324 | 0.0705 | 0.0376 |
| 1 | 112.866 | 99.23154 | 70.48623 | 6.023772 | 6.84123 | 6.035563 | 0.2698 | 0.1070 | 0.0065 | 0.0223 |

As shown in Tables 5 and 6, it was confirmed that the lysate (Table 5) and the fermentation product (Table 6) of Lactobacillus sakei LGHNH22021 strain of the present disclosure inhibited the melanin synthesis in a concentration-dependent manner, and they have the more remarkable melanin synthesis inhibition effects than the lysate and the fermentation product of the comparative strain KCTC3603.

Accordingly, it was confirmed that Lactobacillus sakei LGHNH22021 strain of the present disclosure has the remarkable skin whitening effects.

### Example 6. Evaluation of Skin Moisturizing Effect of Novel Lactobacillus sakei LGHNH22021 Strain

To verify the skin moisturizing effect of the novel strain, human keratinocyte HaCaT was treated with a negative control (control, DMEM containing 0% serum); the lysate (LS-LG) and the fermentation product (LS-LGF) of Lactobacillus sakei LGHNH22021 strain of the present disclosure; the lysate (LS-TS) and the fermentation product (LS-TSF) of Lactobacillus sakei reference strain (KCTC3603); and a broth at each concentration for 24 hours, followed by RNA extraction.

Subsequently, cDNA was synthesized using 1 μg of RNA, expressions of a hyaluronic acid synthase HAS2 and a moisturizing-related gene aquaporin 3 (AQP3) were examined by real-time PCR. The results are shown in Tables 7 (HAS2) and 8 (AQP3) below.

TABLE 7

| | Relative HAS2 | | | | | | |
|---|---|---|---|---|---|---|---|
| | expression level | | | | p-value | | p-value |
| Concentration | average | | STDEV | | (vs control) | | (LS-LG vs |
| (ppm) | LS-TS | LS-LG | LS-TS | LS-LG | LS-TS | LS-LG | LS-TS) |
| control | 1 | 1 | 0.289993 | 0.221953 | — | — | 0.323 |
| 0.1 | 1.1248 | 1.470191 | 0.115151 | 0.348176 | 0.5345 | 0.0589 | 0.638 |
| 1 | 1.1136 | 1.71436 | 0.323081 | 0.288096 | 0.3545 | 0.049 | 0.0515 |
| 10 | 1.1746 | 1.78562 | 0.215469 | 0.190996 | 0.6319 | 0.010 | 0.0285 |

TABLE 8

| | Relative AQP3 | | | | | | | | | |
|---|---|---|---|---|---|---|---|---|---|---|
| | expression level | | | | | | p-value | | | p-value |
| Concentration | average | | | STDEV | | | (vs control) | | | (LS-LGF vs |
| (%) | Broth | LS-TSF | LS-LGF | Broth | LS-TSF | LS-LGF | Broth | LS-TSF | LS-LGF | LS-TSF) |
| control | 1 | 1 | 1 | 0.195 | 0.143 | 0.159 | — | — | — | 0.863 |
| 0.2 | 0.825 | 0.865 | 1.283 | 0.271 | 0.12 | 0.262 | 0.283 | 0.595 | 0.301 | 0.202 |
| 1 | 0.814 | 0.941 | 1.173 | 0.294 | 0.157 | 0.185 | 0.132 | 0.157 | 0.030 | 0.695 |

As shown in Table 7, it was confirmed that the lysate of Lactobacillus sakei LGHNH22021 strain of the present disclosure increased the expression of hyaluronic acid synthase HAS2 in a concentration-dependent manner, as compared to the known Lactobacillus sakei comparative strain KCTC3603. In other words, it has a more remarkable effect of increasing the hyaluronic acid production than the lysate of the comparative strain KCTC3603.

Further, as shown in Table 8, it was confirmed that the fermentation product of Lactobacillus sakei LGHNH22021 strain of the present disclosure increased the expression of water transport membrane protein aquaporin 3 in a concentration-dependent manner, as compared to the known Lactobacillus sakei comparative strain KCTC3603. In other words, it has a more remarkable effect of increasing the expression of aquaporin 3 than the fermentation product of the comparative strain KCTC3603.

Accordingly, it was confirmed that Lactobacillus sakei LGHNH22021 strain of the present disclosure has the remarkable moisturizing effects.

### Example 7. Evaluation of Skin Soothing and Anti-Inflammatory Effects of Novel Lactobacillus sakei LGHNH22021 Strain

To verify the skin soothing and anti-inflammatory effects of the novel strain, mouse macrophage cell line Raw264.7 was treated with a negative control (control, serum-free culture medium); the lysate (LS-LG) and the fermentation product (LS-LGF) of Lactobacillus sakei LGHNH22021 strain of the present disclosure; the lysate (LS-TS) and the fermentation product (LS-TSF) of Lactobacillus sakei reference strain (KCTC3603); and a broth at each concentration for 1 hour, followed by treatment with 0.5 ppm of LPS for 24 hours.

Subsequently, 4% Griess reagent was added to each cell culture medium and then allowed to react at room temperature for 10 minutes. Thereafter, absorbance at 540 nm was measured using a microplate reader. The NO production amount was measured using a NO standard curve, and expressed as a NO production inhibition rate with respect to the negative control group. The results are shown in Tables 9 and 10 below.

TABLE 9

| | | NO production | | | | | | |
|---|---|---|---|---|---|---|---|---|
| | | inhibition rate (%) | | | | p-value | | p-value |
| Concentration | | average | | STDEV | | (vs control) | | (LS-LG vs |
| (ppm) | | LS-TS | LS-LG | LS-TS | LS-LG | LS-TS | LS-LG | LS-TS) |
| LPS | 0 | 0 | 0 | 0.231976 | 0.329715 | — | — | — |
| 0.5 | 0.1 | 2.79516 | 17.61521 | 0.093588 | 1.397613 | 0.210 | 0.047 | 0.013 |
| | 1 | 8.535895 | 25.22114 | 0.499311 | 8.318051 | 0.182 | 0.006 | 0.026 |
| | 10 | 9.180155 | 38.53066 | 1.792564 | 6.906036 | 0.101 | 0.003 | 0.044 |

TABLE 10

| | | NO production | | | | | | | | | |
|---|---|---|---|---|---|---|---|---|---|---|---|
| | | inhibition rate (%) | | | | | | p-value | | | p-value |
| Concentration | | average | | | STDEV | | | (vs control) | | | (LS-LGF vs |
| (%) | | Broth | LS-TSF | LS-LGF | Broth | LS-TSF | LS-LGF | Broth | LS-TSF | LS-LGF | LS-TSF) |
| LPS | control | 0 | 0 | 0 | 1.976435 | 1.03524 | 1.715329 | — | — | — | |
| 0.5 | 0.04 | 9.516753 | 10.6254 | 15.21496 | 3.1235 | 5.3145 | 6.129343 | 0.9495 | 0.0830 | 0.0296 | 0.526 |
| (ppm) | 0.2 | 10.67052 | 12.46825 | 19.35452 | 5.370654 | 5.67415 | 5.122205 | 0.1523 | 0.1094 | 0.0035 | 0.0830 |
| | 1 | 8.634945 | 15.30456 | 24.91176 | 4.9889 | 5.31685 | 3.556268 | 0.2393 | 0.0522 | 0.0023 | 0.0256 |

As shown in Tables 9 and 10, it was confirmed that the lysate (Table 9) and the fermentation product (Table 10) of Lactobacillus sakei LGHNH22021 strain of the present disclosure inhibited the NO production in a concentration-dependent manner, and they had the more remarkable effect of inhibiting the NO production than the lysate and the fermentation product of the comparative strain KCTC3603.

Accordingly, it was confirmed that Lactobacillus sakei LGHNH22021 strain of the present disclosure has the remarkable skin soothing and inflammation improvement effects.

### Example 8. Evaluation of Microbiome Improvement Effects of Novel Lactobacillus sakei LGHNH22021 Strain

The microbiome improvement effect (in vitro probiotic ability) was verified using the fermentation product of the novel strain, and the microbiome improvement effects were compared by measuring changes in the growth rates of beneficial bacteria and harmful bacteria by the control group and the fermentation product of the comparative strain (fermentation product of Lactobacillus sakei reference strain (KCTC3603)).

Among the normal skin microbiota, Staphylococcus epidermidis (S. epidermidis) was selected as a representative beneficial bacterium, and Staphylococcus aureus (S. aureus) was selected as a representative harmful bacterium.

In detail, a sterile liquid medium was prepared using commercially available tryptic soy broth (TSB) medium. S. epidermidis preserved in a glycerol solution or S. aureus preserved in a glycerol solution was inoculated once to twice into the sterile liquid TSB medium, and pre-cultured at 30° C. to 37° C. for 6 hours to 18 hours. To sterile liquid TSB medium, a negative control (control, serum-free culture medium); the fermentation product (LS-LGF) of Lactobacillus sakei LGHNH22021 strain of the present disclosure; the fermentation product (LS-TSF) of Lactobacillus sakei reference strain (KCTC3603); and broth were each added at 1%, and the pre-culture solution was inoculated and cultured at 30° C. to 37° C. for 6 hours to 18 hours. After culturing, the absorbance immediately after inoculation and the absorbance during the stationary phase of growth were measured, and the ratio of the values (growth rate) was used as a statistical variable. The growth rate of the experimental group treated with the sample was compared with that of the control group treated with no sample to determine whether the sample had a significant effect on the growth of bacteria. The evaluation was conducted three times to six times or more, the mean value and standard deviation of the growth rate of the sample were calculated multiple times, and then significance was determined through t-test. The results are shown in Tables 11 and 12 below.

TABLE 11

| | Growth rate (% of control) | | | |
|---|---|---|---|---|
| | | | p-value | p-value (LS-TSF |
| sample | Average | STDEV | (vs control) | vs LS-LGF) |
| control | 100 | 1.367 | — | — |
| Broth | 106 | 1.45 | 0.034 | — |
| LS-TSF | 105.4 | 1.23 | 0.787 | — |
| LS-LGF | 110.632 | 1.014 | 0.002 | 0.026 |

TABLE 12

| | Growth rate (% of control) | | | |
|---|---|---|---|---|
| | | | p-value | p-value (LS-TSF |
| sample | Average | STDEV | (vs control) | vs LS-LGF) |
| control | 100 | 1.57 | — | — |
| Broth | 106 | 2.413 | 0.487 | — |
| LS-TSF | 108.16 | 3.65 | 0.111 | — |
| LS-LGF | 93.74063 | 0.697 | 0.032 | 0.014 |

As shown in Table 11, it was confirmed that the fermentation product of Lactobacillus sakei LGHNH22021 strain of the present disclosure promoted the growth of the beneficial bacterium Staphylococcus epidermidis, as compared to the known Lactobacillus sakei comparative strain KCTC3603. In other words, it has a more remarkable effect of promoting the growth of the beneficial skin bacteria than the lysate of the comparative strain KCTC3603.

Further, as shown in Table 12, it was confirmed that the fermentation product of Lactobacillus sakei LGHNH22021 strain of the present disclosure inhibited the growth of the harmful bacterium Staphylococcus aureus, as compared to the known Lactobacillus sakei comparative strain KCTC3603. In other words, it has a more remarkable effect of inhibiting the growth of the harmful skin bacteria than the lysate of the comparative strain KCTC3603.

Accordingly, it was confirmed that Lactobacillus sakei LGHNH22021 strain of the present disclosure has the remarkable microbiome improvement effects (skin microbiota improvement effects).

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

### ACCESSION NUMBER

Name of Depository Institution: Korea Research Institute of Bioscience and Biotechnology
Accession No: KCTC15087BP
Date of deposit: 2022 Sep. 20

1. A Lactobacillus sakei LGHNH22021 strain deposited with Accession No. KCTC 15087BP.

2. A method for improving in a patient at least one condition selected from the group consisting of anti-aging, wrinkle improvement, elasticity enhancement, skin regeneration, skin whitening, skin moisturization, skin soothing, anti-inflammation, and skin microbiome enhancement, wherein said method comprises administering to said patient an effective amount of the strain according to claim 1.

3. The strain according to claim 1, wherein the strain is derived from Achillea millefolium.

4. A cosmetic composition for skin improvement which comprises at least one member selected from the group consisting of the strain of claim 1, a culture product of said strain, a fermentation product of said strain, a culture medium of said strain, an extract of said strain, and a crushed product of said strain.

5. A method for improving in a patient at least one condition selected from the group consisting of anti-aging, wrinkle improvement, elasticity enhancement, skin regeneration, skin whitening, skin moisturization, skin soothing, anti-inflammation, and skin microbiome enhancement, wherein said method comprises administering to said patient an effective amount of the cosmetic composition according to claim 4.

6. A pharmaceutical composition for skin improvement which comprises at least one member selected from the group consisting of the strain of claim 1, a culture product of said strain, a fermentation product of said strain, a culture medium of said strain, an extract of said strain, and a crushed product of said strain.

7. A food composition for skin improvement which comprises at least one member selected from the group consisting of the strain of claim 1, a culture product of said strain, a fermentation product of said strain, a culture medium of said strain, an extract of said strain, and a crushed product of said strain.

8. A method for promoting the proliferation of Staphylococcus epidermidis, beneficial skin bacteria in a patient which method comprises administering to said patient at least one member selected from the group consisting of the strain of claim 1, a culture product of said strain, a fermentation product of said strain, a culture medium of said strain, an extract of said strain, and a crushed product of said strain.

9. A method for inhibiting the proliferation of Staphylococcus aureus, harmful skin bacteria in a patient, which method comprises administering to said patient at least one member selected from the group consisting of the strain of claim 1, a culture product of said strain, a fermentation product of said strain, a culture medium of said strain, an extract of said strain, and a crushed product of said strain.

10. A method for preparing a fermentation product, comprising fermenting the strain of claim 1.

11. A method for preparing a lysate, comprising crushing the strain of claim 1.
